(19) United States
(12) Patent Application Publication
(10) Pub. No.: US 2026/0334774 A1
(43) Pub. Date: Oct. 29, 2026

(54) PATIENT SUITABILITY ASSESSMENT FOR OPHTHALMIC PROCEDURES

(71) Applicant: Alcon Inc., Fribourg (CH)

(72) Inventors: George Hunter Pettit, Fort Worth, TX (US); John Alfred Campin, St. Cloud, FL (US)

(21) Appl. No.: 19/660,924

(22) Filed: Apr. 28, 2026

Related U.S. Application Data
(60) Provisional application No. 63/796,632, filed on Apr. 29, 2025.

(51) Int. Cl. A61B 3/00; A61B 3/10

(52) U.S. Cl. CPC A61B 3/0025; A61B 3/0033; A61B 3/0041; A61B 3/10

(57) ABSTRACT

The present disclosure generally relates to a system and method that includes acquiring, using an ophthalmic imaging device, diagnostic image(s) of an eye of a patient. The method further includes applying image processing algorithm(s) to the diagnostic image(s) to determine measurements corresponding to anatomical parameters of the eye. The method further includes accessing patient suitability information for a prospective ophthalmic procedure. The patient suitability information includes a set of values corresponding to a subset of the plurality of anatomical parameters. The method further includes generating a suitability assessment for performing the prospective ophthalmic procedure on the eye. Generating the suitability assessment comprises comparing a subset of the measurements, corresponding to the subset of the anatomical parameters, with the set of values of the patient suitability information. The method further includes presenting information from the suitability assessment using a patient-oriented display.

### INTRODUCTION

Patients considering undergoing an ophthalmic procedure, such as cataract surgery or refractive surgery, are typically subjected to comprehensive diagnostic imaging of their eye(s) in a clinical setting to inform treatment recommendations by surgeons or other medical providers. Many ophthalmic imaging modalities, such as optical coherence tomography (OCT), fluorescein angiography, and fundus photography, generate extremely detailed images that require specialized knowledge to interpret correctly. However, presenting the diagnostic imaging or derived parameters directly to patients can pose challenges due to the complexity of the data and the patients' varying levels of health literacy. Patients lacking medical training may struggle to understand the significance of the diagnostic images, leading to confusion, anxiety, or misinterpretation of their condition.

While it is important for the patients to understand certain diagnostic findings and related implications when deciding an appropriate treatment approach, ophthalmic imaging devices are typically configured to display information for interpretation primarily by the medical provider. As a result, the diagnostic imaging may not facilitate the patient’s understanding of important information, often include numerous parameters that are not readily understood by the patient or not relevant to their treatment decision.

### SUMMARY

The present disclosure generally relates to techniques for determining a suitability for performing a prospective ophthalmic procedure on an eye of a patient, and intelligently presenting information about the patient’s suitability using a patient-oriented display. In some aspects, the method includes acquiring, using an ophthalmic imaging device, diagnostic image(s) of an eye of a patient. The method further includes applying image processing algorithm(s) to the diagnostic image(s) to determine measurements corresponding to anatomical parameters of the eye. The method further includes accessing patient suitability information for a prospective ophthalmic procedure. The patient suitability information includes a set of values corresponding to a subset of the plurality of anatomical parameters. The method further includes generating a suitability assessment for performing the prospective ophthalmic procedure on the eye. Generating the suitability assessment comprises comparing a subset of the measurements, corresponding to the subset of the anatomical parameters, with the set of values of the patient suitability information. The method further includes presenting information from the suitability assessment using a patient-oriented display.

The techniques can provide select diagnostic images and/or parameters that are relevant to the prospective ophthalmic procedure to be displayed to the patient, and these parameters to be visually referenced to portions of the diagnostic images, such as highlighting anatomical structures within the diagnostic images, adding annotations, and so forth. Further, different treatment options may be intuitively presented to the patient, e.g., generating simulated views of a reference image that account for performing the prospective ophthalmic procedure according to different parameters. In this way, the techniques can improve the patient’s understanding regarding their medical condition and potential treatments and expected outcomes, supporting better communication between the patient and the medical provider, and resulting in improved decision-making for treatment and better outcomes. Although ophthalmic procedures are discussed as examples of procedures that may benefit from the described aspects, the advantages of the surgical devices and systems described herein may benefit procedures in other clinical and/or surgical environments as well.

### BRIEF DESCRIPTION OF THE DRAWINGS

So that the manner in which the above recited features of the present disclosure can be understood in detail, a more particular description of the disclosure, briefly summarized above, may be had by reference to aspects, some of which are illustrated in the appended drawings. It is to be noted, however, that the appended drawings illustrate only exemplary aspects and are therefore not to be considered limiting of its scope, and may admit to other equally effective aspects.

FIG. 1 illustrates a perspective view of an example ophthalmic imaging device, according to one or more aspects.

FIG. 2A illustrates a side view of a patient undergoing an example diagnostic examination, according to one or more aspects.

FIG. 2B illustrates a top view of the example diagnostic examination of FIG. 2A, according to one or more aspects.

FIG. 3 illustrates a block diagram of an example ophthalmic system, according to one or more aspects.

FIG. 4 is a method of generating a suitability assessment for a prospective ophthalmic procedure, according to one or more aspects.

FIG. 5 illustrates displaying an example suitability assessment using a patient-oriented display, according to one or more aspects.

FIG. 6 illustrates views of a reference image simulating views of an eye after performing a prospective ophthalmic procedure according to different parameters, according to one or more aspects.

To facilitate understanding, identical reference numerals have been used, where possible, to designate identical elements that are common to the figures. It is contemplated that elements and features of one aspect may be beneficially incorporated in other aspects without further recitation.

### DETAILED DESCRIPTION

The present disclosure generally relates to techniques for determining a suitability for performing a prospective ophthalmic procedure on an eye of a patient, and intelligently presenting information about the patient’s suitability using a patient-oriented display.

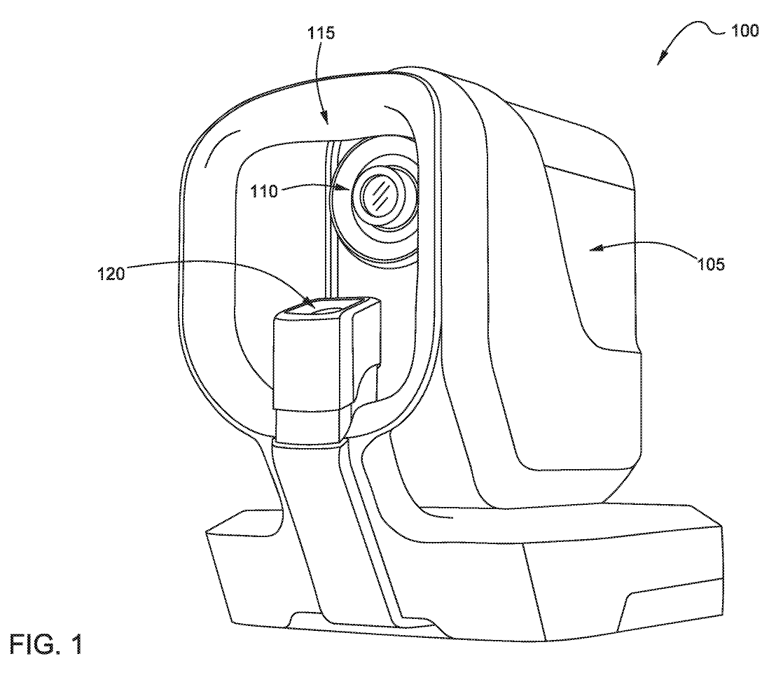

FIG. 1 illustrates a perspective view of an example ophthalmic imaging device 100, according to one or more aspects. FIG. 1 depicts a first side of the ophthalmic imaging device 100 that faces the patient during a diagnostic procedure, allowing the eye(s) of the patient to be suitably positioned for the procedure. As shown in FIG. 1, the ophthalmic imaging device 100 is an upright hyperparallel optical coherence tomography (HP-OCT) device and comprises a headrest 115 for positioning a forehead of the patient, a chin rest 120 for positioning a chin of the patient, and a measurement module 105. The ophthalmic imaging device 100 further includes an optical system 110 (for example, disposed within a housing of the measurement module 105) that aligns with the eye(s) of the patient when his or her head is positioned against the headrest 115 and/or the chin rest 120, enabling diagnostic images of the eye(s) to be acquired. In some embodiments, the optical system 110 includes a succession of optical components, such as one or more sensors, detectors, light sources, lenses, mirrors, beam splitters, beam combiners, projection systems, reflecting prisms, filters and thin films, fiber optics, and/or the like, for performing one or more diagnostic functions of the ophthalmic imaging device 100. The ophthalmic imaging device 100 may further include one or more adjustment mechanisms that adjust the relative positioning of the optical system 110 and the eye(s) of the patient to improve aligned of the eye(s) with the optical system 110.

While the ophthalmic imaging device 100 is discussed primarily in terms of a HP-OCT device, other types of ophthalmic imaging devices and optometric devices are also contemplated. Some non-limiting examples include an optical biometer, an OCT instrument such as a swept source-OCT (SS-OCT) biometer, an OCT retina and/or anterior segment imaging system, other low-coherence interferometry instrument, a conventional or wide-angle fundus camera for taking images of the eye, a digital fundus camera, an instrument for fundus autofluorescence (AF) and/or multispectral imaging, a keratometer, an autorefractor, a topography measurement device such as a corneal topography device, a device for tear film assessment, an ultrasound device such as a B-scan ultrasound device, or combinations thereof. Further, the ophthalmic imaging device(s) or optometric device(s) need not be exclusive in function, but may be integrated into other devices or systems that are used by the patient and/or the medical provider (e.g., a body-worn headset providing a display functionality to the patient).

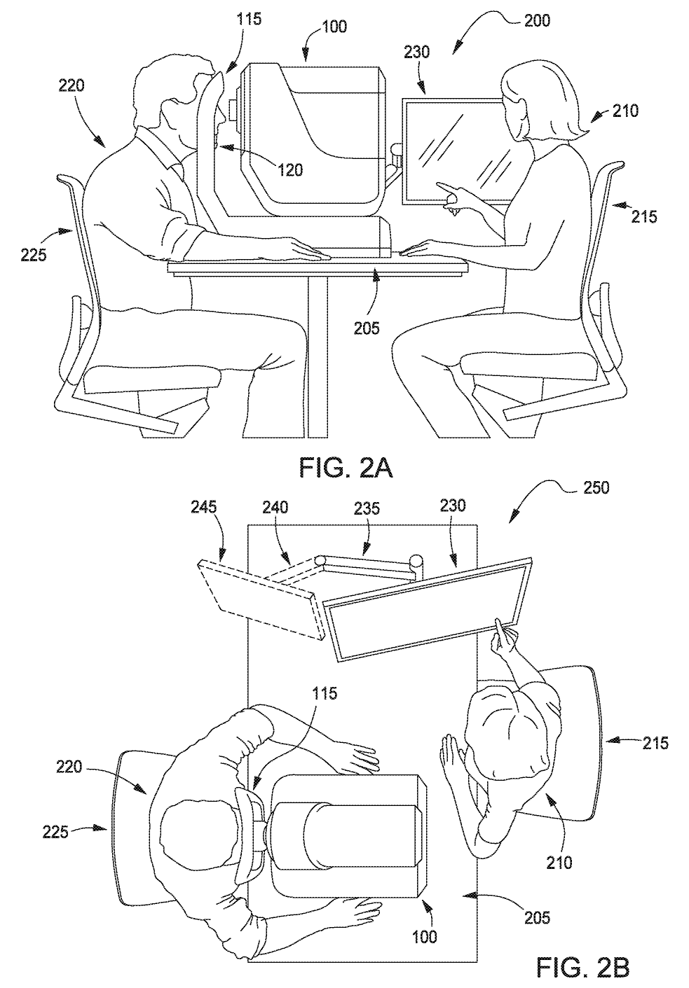

FIG. 2A illustrates a side view 200, and FIG. 2B illustrates a top view 250, respectively, of a patient undergoing an example diagnostic examination, according to one or more aspects. The features illustrated in the side view 200 and the top view 250 may be used in conjunction with other embodiments described herein.

The ophthalmic imaging device 100 is disposed on a table 205. A patient 220 is seated in a first chair 225 on one side of the table 205, and a medical provider 210 (e.g., a technician, clinician, or a surgeon) is seated in a second chair 215 on the opposing side of the table 205. The head of the patient 220 is positioned relative to the ophthalmic imaging device 100 to allow diagnostic imaging of his eye(s). As shown, the patient 220 rests his forehead against the headrest 115 and his chin on the chin rest 120 to align his eye(s) with the optical system 110. The headrest 115, the chin rest 120, and/or the optical system 110 may be adjusted to improve alignment of the optical system 110 with the eye(s) of the patient 220.

One or more displays 230, 245 are communicatively connected with the ophthalmic imaging device 100 and are supported by the table 205. As shown, the displays 230, 245 are connected to respective articulating arms 235, 240, which may be connected to a base or other support structure that is disposed on, or connected to, the table 205.

In some embodiments, two separate displays 230, 245 are provided: a first, provider-facing display 230 that is oriented toward the medical provider 210, and a second, patient-facing display 245 that is oriented toward the patient 220. In other embodiments, a single display 230 is provided that is repositionable between a provider-facing orientation and a patient-facing orientation, e.g., by manipulating the articulating arm 235 and/or pivoting the display 230 relative to the articulating arm 235. In some embodiments, the content displayed on the display 230 may be different depending on the current orientation. In some embodiments, the displays 230, 245 may be implemented as touchscreens that are capable of receiving inputs from the medical provider 210 and/or the patient 220.

During the diagnostic examination, the ophthalmic imaging device 100 acquires one or more diagnostic images of the eye(s) of the patient 220, and performs one or more image processing algorithms to determine a plurality of anatomical parameters of the eye(s). The one or more processing algorithms may operate at any level of complexity, and in some embodiments include one or more of the following: an edge detection algorithm, an object localization algorithm, an object detection algorithm, an image segmentation algorithm, a feature detection algorithm, and a feature extraction algorithm. In some embodiments, the one or more image processing algorithms may be used to develop a 3D digital model of the eye(s) of the patient 220. In some embodiments, the one or more image processing algorithms identify a plurality of anatomical structures of the eye, and determine a plurality of measurements corresponding to a plurality of anatomical parameters. Using the example of the HP-OCT device, the ophthalmic imaging device 100 may determine anatomical parameters such as a retinal thickness, retinal nerve fiber layer thickness, macular volume and thickness, ganglion cell complex thickness, choroidal thickness, anterior chamber depth, corneal thickness, angle of the anterior chamber, optic nerve head parameters, and so forth.

Some or all of the diagnostic images acquired by the ophthalmic imaging device 100 (as well as the anatomical parameters derived therefrom) may be presented to the medical provider 210 using a first graphical user interface (GUI) on the provider-facing display 230. In some embodiments, the medical provider 210 may browse and/or manipulate the diagnostic image(s) and/or anatomical parameter(s) by providing inputs to the provider-facing display 230.

As discussed above, the ophthalmic imaging device 100 may be configured to display information for interpretation primarily by the medical provider 210. Thus, in some cases the diagnostic images and anatomical parameters that are displayed on the provider-facing display 230 may be more numerous and/or more complex, which can negatively affect the patient’s comprehension of their condition and the possible treatments.

In some embodiments, a subset of the diagnostic images and/or a subset of the anatomical parameters are displayed on the patient-facing display 245 using a second GUI. As will be discussed in greater detail below, the subset(s) may be selected for display based on factors such as the prospective ophthalmic procedure and an assessment of the patient’s suitability therefor. In many cases, the patient 220 is presented fewer diagnostic images and/or anatomical parameters than the medical provider 210, and those diagnostic images and/or anatomical parameters that are presented have been determined to have a greater relevance to the prospective ophthalmic procedure or to the patient’s suitability.

The second GUI may include additional visual elements that improve the explainability the subset of the diagnostic images and/or the subset of the anatomical parameters for the patient 220. In some embodiments, the second GUI visually references one or more anatomical parameters to corresponding portion(s) of the diagnostic images, e.g., by highlighting the anatomical structure(s) within the at least one diagnostic image, adding annotations, and so forth.

The second GUI may also present information about potential treatments for the patient. In some embodiments, the second GUI displays, based on the plurality of anatomical parameters, a first view of a reference image simulating a view of the eye after performing the prospective ophthalmic procedure. The first view may correspond to performing the prospective ophthalmic procedure according to a first parameter, and a second view of the reference image is displayed simulating a view of the eye after performing the prospective ophthalmic procedure according to a second parameter. For example, when the prospective ophthalmic procedure is a cataract surgical procedure, the first parameter is a non-diffractive intraocular lens (IOL), and the second parameter is a diffractive IOL.

In this way, the techniques can improve the patient’s understanding regarding their medical condition and potential treatments and expected outcomes, supporting better communication between the patient 220 and the medical provider 210, and resulting in improved decision-making for treatment and better outcomes.

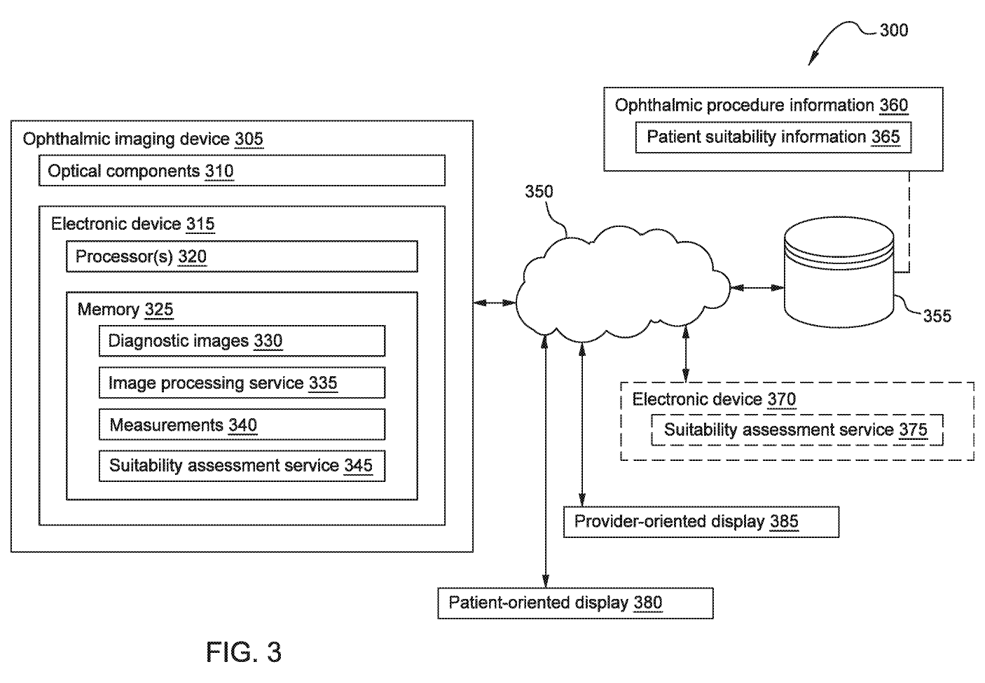

FIG. 3 illustrates a block diagram of an example ophthalmic system 300, according to one or more aspects. The features of the ophthalmic system 300 may be used in conjunction with other embodiments. For example, the ophthalmic system 300 may be used to conduct the diagnostic examination of FIGS. 2A, 2B.

The ophthalmic system 300 comprises an ophthalmic imaging device 305 that is communicatively connected through a network 350, to a database 355 or other computer storage, a patient-oriented display 380, a provider-oriented display 385, and optionally an electronic device 370. The network 350 may have any suitable implementation, such as one or more wide area networks (WANs), one or more local access networks (LANs), or combinations thereof. The network 350 comprises infrastructure for communicative capability, such as conductive cabling, wireless transmission, optical transmission, and so forth. The network 350 may further comprise one or more electronic devices providing network functionality and/or services to the network 350, such as routers, firewalls, switches, gateway computers, edge servers, and so forth.

In some embodiments, the ophthalmic imaging device 305 is the ophthalmic imaging device 100 depicted in FIG. 1, e.g., HP-OCT device, although other types of ophthalmic imaging devices and optometric devices are also contemplated. The ophthalmic imaging device 305 comprises a plurality of optical components 310, e.g., the optical components of the optical system 110. The ophthalmic imaging device 305 further comprises an electronic device 315.

As used herein, an “electronic device” generally refers to any device having electronic circuitry that provides a processing or computing capability, and that implements logic and/or executes program code to perform various operations that collectively define the functionality of the electronic device. The functionality of the electronic device includes a communicative capability with one or more other electronic devices, e.g., when connected to a same network. An electronic device may be implemented with any suitable form factor, whether relatively static in nature (e.g., mainframe, computer terminal, server, kiosk, workstation) or mobile (e.g., laptop computer, tablet, handheld, smart phone, wearable device). The communicative capability between electronic devices may be achieved using any suitable techniques, such as conductive cabling, wireless transmission, optical transmission, and so forth.

As shown, the electronic device 315 comprises one or more processors 320 and a memory 325. The one or more processors 320 include any electronic circuitry, including, but not limited to one or a combination of microprocessors, microcontrollers, application-specific integrated circuits (ASIC), application-specific instruction set processors (ASIP), and/or state machines, that is communicatively coupled to the memory and controls the operation of the system. In some aspects, the electronic circuitry is configured to perform any of the functions described herein. Further, the one or more processors 320 are not limited to a single processing device and may encompass multiple processing devices.

The one or more processors 320 may include other hardware that operates software to control and process information. In some aspects, the one or more processors 320 execute software stored in the memory to perform any of the functions described herein. The one or more processors 320 control the operation and administration of the electronic device 315 by processing information (e.g., information received from input devices and/or communicatively coupled electronic devices).

The memory 325 may store, either permanently or temporarily, data, operational software, or other information for the one or more processors 320. The memory 325 may include any one or a combination of volatile or non-volatile local or remote devices suitable for storing information. For example, the memory 325 may include random-access memory (RAM), read-only memory (ROM), magnetic storage devices, optical storage devices, or any other suitable information storage device or a combination of these devices. The software represents any suitable set of instructions, logic, or code embodied in a computer-readable storage medium. For example, the software may be embodied in the memory 325, a disk, a CD, or a flash drive. In particular embodiments, the software may include an application executable by the one or more processors 320 to perform one or more of the functions described herein.

The memory 325 stores a plurality of diagnostic images 330 that are acquired by the ophthalmic imaging device 305 from the plurality of optical components 310. The plurality of diagnostic images 330 may include any suitable number of images with any suitable formatting.

The memory 325 further stores an image processing service 335 that applies one or more image processing algorithms to the plurality of diagnostic images 330. The one or more image processing algorithms may include one or more of the following: an edge detection algorithm, an object localization algorithm, an object detection algorithm, an image segmentation algorithm, a feature detection algorithm, a feature extraction algorithm, and so forth.

In some embodiments, the one or more image processing algorithms are operated to identify a plurality of anatomical structures of the eye(s) of the patient, and to determine a plurality of measurements 340 corresponding to a plurality of anatomical parameters. Using the example of the HP-OCT device, the plurality of anatomical parameters may include a retinal thickness, retinal nerve fiber layer thickness, macular volume and thickness, ganglion cell complex thickness, choroidal thickness, anterior chamber depth, corneal thickness, angle of the anterior chamber, optic nerve head parameters, and so forth.

The memory 325 further stores a suitability assessment service 345 that determines, based on some or all of the plurality of measurements 340, the patient’s suitability to undergo a prospective ophthalmic procedure. In some embodiments, the suitability assessment service 345 receives an indication of the prospective ophthalmic procedure (e.g., provided by user input), and using the prospective ophthalmic procedure accesses patient suitability information 365 against which the plurality of measurements 340 are compared. In other embodiments, the suitability assessment service 345 uses the plurality of measurements 340 to identify one or more prospective ophthalmic procedures that may be suitable for the patient.

In some embodiments, the database 355 stores ophthalmic procedure information 360 corresponding to one or more ophthalmic procedures. The ophthalmic procedure information 360 may include information describing the steps or other aspects of performing each ophthalmic procedure, as well as the patient suitability information 365 that indicates one or more anatomical parameters and one or more values (or ranges of values) thereof. The one or more values may include a range of acceptable values of the anatomical parameter, a maximum value, a minimum value, a distribution of values for a reference population (or characteristics of the distribution such as a mean and variance), and so forth.

The patient suitability information 365 may have any suitable formatting, such as structured data or semi-structured data of an object. For example, the patient suitability information 365 may include a plurality of records (or entries) corresponding to a plurality of ophthalmic procedures, where each record includes fields for the one or more anatomical parameters and for the one or more values (or ranges of values). In some embodiments, the ophthalmic procedure information 360 includes a plurality of anatomical parameters (e.g., representing a comprehensive set of anatomical parameters for imaging the eye(s)), and the one or more anatomical parameters for a particular ophthalmic procedure is a subset of the plurality of anatomical parameters having greater relevance to the ophthalmic procedure. In some embodiments, the patient suitability information 365 for a procedure includes fields for the subset (e.g., those anatomical parameters having greater than a threshold relevance) and corresponding values. In other embodiments, the patient suitability information 365 includes fields for the plurality of anatomical procedures, the relevance values for the anatomical procedures, and the corresponding values, such that the one or more anatomical parameters may be selected from the set according to a threshold relevance, a number of the most relevant anatomical parameters (e.g., selecting top-three, top-five), and so forth.

Continuing the example of the HP-OCT device, and using cataract surgery as an example of the prospective ophthalmic procedure, the subset of the plurality of anatomical parameters may include anterior chamber depth, corneal thickness (e.g., central corneal thickness), angle of the anterior chamber, macular volume and thickness, and optic nerve head parameters. Thus, the patient suitability information 365 for cataract surgery may include fields for the subset of the plurality of anatomical parameters and/or values thereof, and other anatomical parameters (e.g., retinal thickness, retinal nerve fiber layer thickness, ganglion cell complex thickness, choroidal thickness) may be omitted.

The suitability assessment service 345 accesses the patient suitability information 365 for the prospective ophthalmic procedure, and generates a suitability assessment for performing the prospective ophthalmic procedure on the eye(s). Generating the suitability assessment comprises comparing a subset of the plurality of measurements 340, corresponding to the subset of the plurality of anatomical parameters, with the set of values (or ranges) of the patient suitability information 365.

In some embodiments, the suitability assessment service 345 determines, for each anatomical parameter of the subset, whether the corresponding measurement is within a range that is defined by the value(s) of the patient suitability information 365. For example, the suitability assessment service 345 may determine whether the measurement is within the range as expressly defined in the patient suitability information 365, may determine whether the measurement is less than a maximum value of the patient suitability information 365, and so forth.

The suitability assessment service 345 generates the suitability assessment based on the anatomical parameters of the subset for the prospective ophthalmic procedure. The suitability assessment may have any suitable formatting. For example, the suitability assessment may report, for each anatomical parameter of the subset, whether the measurement is within the corresponding range, may include a binary classification of whether the patient is suitable or not suitable (e.g., not suitable if any of the anatomical parameters are out-of-range), may generate a numerical score (e.g., 70% for seven of ten anatomical parameters having within-range measurements), and so forth. In some embodiments, the suitability assessment service 345 compares the numerical score with a threshold to determine whether the patient is suitable or not suitable, which may be shared by the prospective ophthalmic procedures, or may differ for different prospective ophthalmic procedures (e.g., a higher threshold applied for a procedure having a higher associated risk).

The suitability assessment service 345 presents some or all of the suitability assessment to the medical provider 210 using the provider-oriented display 385 (e.g., the provider-facing display 230 of FIGS. 2A, 2B), and/or to the patient 220 using the patient-oriented display 380 (e.g., the patient-facing display 245 of FIGS. 2A, 2B). In some embodiments, the suitability assessment service 345 presents all of the suitability assessment to the medical provider 210, and portions of the suitability assessment to the patient 220. Presenting the information from the suitability assessment comprises displaying at least one diagnostic image of the one or more diagnostic images 330, and visually referencing one or more anatomical parameters of the subset of the plurality of anatomical parameters to a corresponding one or more portions of the at least one diagnostic image.

In some embodiments, the suitability assessment service 345 selects the at least one diagnostic image based on whether the diagnostic images depict anatomical structures of the eye that correspond to the one or more anatomical parameters of the subset. For example, when corneal thickness is included in the subset, the suitability assessment service 345 may select diagnostic image(s) that depict the anterior surface and posterior surface of the cornea. In some embodiments, when the plurality of diagnostic images 330 includes a 3D model of the eye, the suitability assessment service 345 may select a 2D projection of the 3D model (e.g., corresponding to a particular orientation of the 3D model) that depict the anatomical structures. The suitability assessment service 345 may select a number of diagnostic images that illustrate some or all of the one or more anatomical parameters of the subset, improving the explainability of the suitability assessment to the patient 220. For example, the suitability assessment service 345 may select a minimum number of diagnostic images that collectively depict all of the one or more anatomical parameters of the subset, a predefined number of diagnostic images that depict the most relevant anatomical parameters of the subset, and so forth. In this way, the suitability assessment service 345 is capable of presenting to the patient 220 those anatomical parameter(s) that are relevant to the prospective ophthalmic procedure, independent of the suitability or non-suitability of the patient 220.

In other embodiments, however, the suitability assessment service 345 is capable of presenting to the patient 220 those anatomical parameter(s) that are relevant to the suitability or non-suitability of the patient 220. The suitability assessment service 345 may select one or more diagnostic images as being illustrative of anatomical parameter(s) having particular relevance to the suitability determination performed by the suitability assessment service 345. For example, assume that the corneal thickness of the patient 220 is outside the acceptable range for cataract surgery, causing the patient 220 to be determined not suitable for the procedure. The suitability assessment service 345 may select a diagnostic image that depicts the anterior surface and posterior surface of the cornea, such that the (disqualifying) anatomical parameter may be displayed to the patient 220.

The suitability assessment service 345 visually references one or more anatomical parameters of the subset to a corresponding one or more portions of the selected diagnostic image(s). In some embodiments, visually referencing the one or more anatomical parameters comprises one or both of highlighting the anatomical structure(s) for the anatomical parameters within the diagnostic image(s), and adding annotations.

In some embodiments, the suitability assessment service 345 supports an interactive display permitting the patient 220 to provide inputs responsive to the displayed suitability assessment. For example, the suitability assessment service 345 may provide graphical elements indicating that additional information is available for specific elements displayed in the suitability assessment. The suitability assessment service 345 may access the ophthalmic procedure information 360 to display additional information for the presented information. In this way, the suitability assessment service 345 can adapt the display based on specific prompts from the patient 220 (e.g., to provide further explanation to the patient 220) or on increased interest in any particular procedure or variant.

Although various components are shown in the memory 325, in some embodiments, some or all of the diagnostic images 330, the image processing service 335, and/or the plurality of measurements 340 may be implemented in a remote electronic device (e.g., the database 355 or the electronic device 370). For example, some or all of the functionality of the suitability assessment service 345 may be implemented as suitability assessment service 375 within the electronic device 370. This can be beneficial for implementations of the ophthalmic imaging device 305 that have relatively less computing resources (e.g., less storage, slower processor(s) 320), as some or all of the processing and/or storage may be offloaded to provide suitable performance levels and/or reduce costs. Further, in other embodiments, the ophthalmic procedure information 360 with the patient suitability information 365 may be implemented locally in the memory of the ophthalmic imaging device 305, such that accessing resources through the network 350 is not necessary.

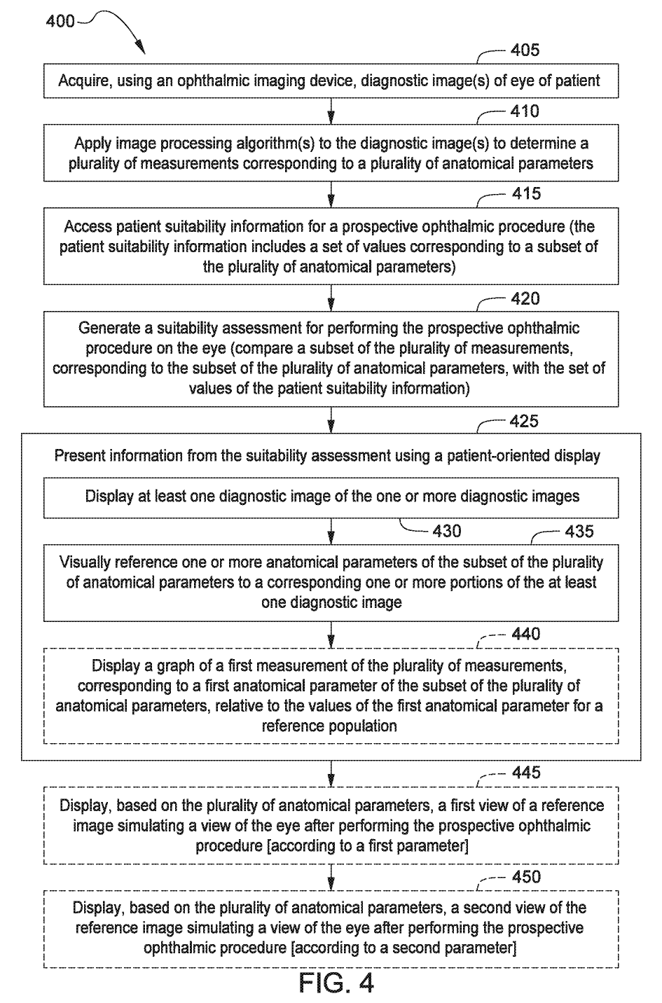

FIG. 4 is a method 400 of generating a suitability assessment for a prospective ophthalmic procedure, according to one or more aspects. The method 400 may be used in conjunction with other embodiments, e.g., using the image processing service 335 and suitability assessment service 345 of FIG. 3.

The method 400 begins at block 405, where the ophthalmic imaging device 305 acquires one or more diagnostic images 330 of an eye of the patient 220. At block 410, the image processing service 335 applies one or more image processing algorithms to the one or more diagnostic images 330 to determine a plurality of measurements 340 corresponding to a plurality of anatomical parameters of the eye. In some embodiments, the one or more image processing algorithms include one or more of an edge detection algorithm, an object localization algorithm, an object detection algorithm, an image segmentation algorithm, a feature detection algorithm, and a feature extraction algorithm. In some embodiments, the one or more image processing algorithms identify a plurality of anatomical structures of the eye, and the image processing service 335 determines the plurality of measurements based on the plurality of anatomical structures.

At block 415, the suitability assessment service 345 accesses patient suitability information 365 for a prospective ophthalmic procedure, where the patient suitability information 365 includes a set of values corresponding to a subset of the plurality of anatomical parameters. In some embodiments, the prospective ophthalmic procedure is identified by a user input to the ophthalmic imaging device 305. In other embodiments, the ophthalmic imaging device 305 selects the prospective ophthalmic procedure as part of assessing the suitability of a plurality of ophthalmic procedures for the patient 220 (e.g., selected according to a predefined sequence). The set of values may define ranges of acceptable values for the respective anatomical parameters.

At block 420, the suitability assessment service 345 generates a suitability assessment for performing the prospective ophthalmic procedure on the eye. In some embodiments, generating the suitability assessment comprises comparing a subset of the plurality of measurements 340, corresponding to the subset of the plurality of anatomical parameters, with the set of values of the patient suitability information 365. In some embodiments, the suitability assessment includes a report of whether each measurement of the anatomical parameters of the subset falls within the corresponding range of acceptable values, a binary classification of the patient’s suitability, a numerical suitability score, and so forth.

At block 425, the suitability assessment service 345 presents information from the suitability assessment using the patient-oriented display 380. In some embodiments, the suitability assessment service 345 presents less information from the suitability assessment than is displayed using the provider-oriented display 385.

In some embodiments, presenting information from the suitability assessment comprises, at block 430, displaying at least one diagnostic image of the one or more diagnostic images 330. In some embodiments, the suitability assessment service 345 selects the at least one diagnostic image based on a relevance of the diagnostic image to the prospective ophthalmic procedure, and/or to the patient’s suitability for the prospective ophthalmic procedure.

In some embodiments, presenting information from the suitability assessment further comprises, at block 435, visually referencing one or more anatomical parameters of the subset of the plurality of anatomical parameters to a corresponding one or more portions of the at least one diagnostic image. In some embodiments, presenting information from the suitability assessment further comprises, at optional block 440, displaying a graph of a first measurement of the plurality of measurements, corresponding to a first anatomical parameter of the subset of the plurality of anatomical parameters, relative to the values of the first anatomical parameter for a reference population.

In some embodiments, the method 400 further comprises receiving a user input indicating an interaction of the patient with the presented information from the suitability assessment, and displaying, based on the user input, additional information related to the presented information using the patient-oriented display 380. For example, the patient 220 may provide the user input by a touch input to the patient-oriented display 380 (e.g., pressing at or near particular displayed information) or the medical provider 210 may provide the user input responsive to a question of the patient 220 about the displayed information. In some embodiments, the suitability assessment service 345 accesses the ophthalmic procedure information 360 responsive to the user input and provides the additional information about the displayed information.

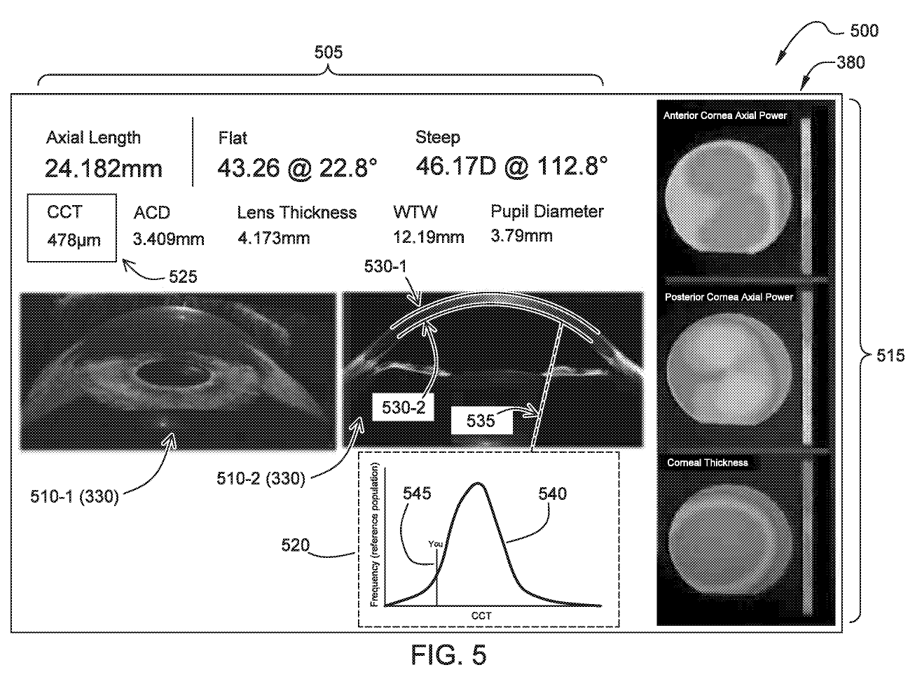

Referring to FIG. 5, which illustrates displaying an example suitability assessment using a patient-oriented display. More specifically, the diagram 500 represents an example display using the second GUI on the patient-oriented display 380.

The second GUI includes a first region 505 displaying a first set of a plurality of anatomical parameters: an axial length of the eye, a flattest corneal power (K), a steepest K, a central corneal thickness (CCT), an anterior chamber depth (ACD), a lens thickness, a white-to-white (WTW) corneal diameter, and a pupil diameter. In some embodiments, the plurality of anatomical parameters that are displayed in the first region 505 may be selected based on the prospective ophthalmic procedure and/or the patient’s suitability for the prospective ophthalmic procedure. The first region 505 further displays values of each of the plurality of anatomical parameters.

The second GUI further includes one or more diagnostic images 510-1, 510-2 arranged below the first region 505. The one or more diagnostic images 510-1, 510-2 are selected from the diagnostic images 330 to the prospective ophthalmic procedure, which may be selected based on a relevance of the one or more diagnostic images 510-1, 510-2 to the prospective ophthalmic procedure and/or the relevance of the suitability of the patient 220 for the prospective ophthalmic procedure.

The second GUI includes a second region 515 to the right of the first region 505 and the one or more diagnostic images 510-1, 510-2. The second region 515 displays a plurality of heat maps for a second set of a plurality of anatomical parameters: an anterior cornea axial power, a posterior cornea axial power, and a corneal thickness. The heat maps provide a 2D (areal) graphical representation of the plurality of anatomical parameters.

The suitability assessment service 345 visually references one or more anatomical parameters of the subset of the plurality of anatomical parameters to a corresponding one or more portions of the at least one diagnostic image. As shown, one anatomical parameter (e.g., CCT) of the first region 505 and the corresponding measurement (e.g., 478 µm) is overlaid in the second GUI with a bounding box 525. The bounding box 525 may be responsive to a user input selecting the anatomical parameter (e.g., a finger press on the corresponding region of the first region 505) or automatically selected by the suitability assessment service 345 (e.g., as a particularly relevant anatomical parameter). In some embodiments, various ones of the anatomical parameters of the first region 505 and/or the second region 515 may be selected in a similar manner. In some embodiments, one or more of the diagnostic images 510-1, 510-2 may be replaced by another diagnostic image 330 based on the selected anatomical parameter.

The anatomical structures corresponding to the anatomical parameter (specifically, the anterior corneal surface 530-1 and the posterior corneal surface 530-2) are highlighted in the diagnostic image 510-2. A leader 535 extends between the posterior corneal surface 530-2 and a graph 520 of the measurement relative to values of the anatomical parameter for a reference population. As shown, the graph 520 includes a plot 540 illustrating a distribution of the reference population values, as well as the position 545 of the measurement relative to the distribution. The graph 520 may include limits such as an acceptable range, a minimum value, a maximum value, and so forth.

Thus, using the second GUI of the patient-oriented display 380, various anatomical parameters that are relevant to the prospective ophthalmic procedure may be visually identified within the plurality of diagnostic images 330, and additional information may presented illustrating the patient’s measurement relative to a range of acceptable values or a reference population, helping the patient 220 to more easily grasp the nature of the anatomical parameters and the patient’s suitability for the prospective ophthalmic procedure.

Returning to FIG. 4, at optional block 445, the suitability assessment service 345 displays, based on the plurality of anatomical parameters, a first view of a reference image simulating a view of the eye after performing the prospective ophthalmic procedure. In some embodiments, the first view corresponds to performing the prospective ophthalmic procedure according to a first parameter. At an optional block 450, the suitability assessment service 345 displays, based on the plurality of anatomical parameters, a second view of the reference image simulating a view of the eye after performing the prospective ophthalmic procedure according to a second parameter. The first view and the second view may be displayed using the patient-oriented display 380, whether as elements within the second GUI or separately.

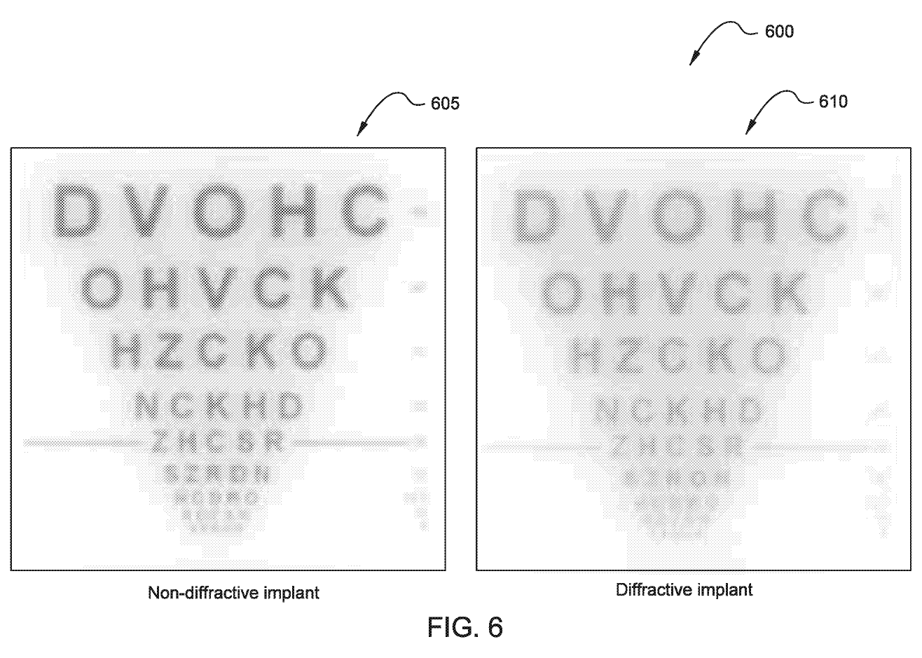

Referring to FIG. 6, which is a diagram 600 illustrating views 605, 610 of a reference image simulating views of an eye after performing a prospective ophthalmic procedure according to different parameters. More specifically, for a cataract surgery in which an IOL would be implanted in the patient’s eye, a first view 605 simulates the patient’s view (using some or all of the plurality of anatomical parameters representing the patient’s eye) through a non-diffractive implant and the second view 610 simulates the patient’s view through a diffractive implant. The reference image is a visual acuity chart (e.g., a logMAR chart) having rows of letters at different sizes, although other images (graphical, photographs) may be used to simulate various aspects of the patient’s vision, such as color vision, contrast sensitivity, stereoacuity, and so forth following the prospective ophthalmic procedure.

Returning to FIG. 4, the method 400 ends following completion of the optional block 450. Thus, the suitability assessment service 345 provides an interface that allows the patient 220 to have a more intuitive understanding of their medical condition and their suitability for a prospective ophthalmic procedure, as well as options for the prospective ophthalmic procedure and the likely outcomes. This supports better communication between the patient 220 and the medical provider 210, and results in improved decision-making for treatment and better overall outcomes.

### EMBODIMENTS

The present disclosure is further directed to the following embodiments which may be combined with any embodiments described herein and/or may be isolated (optional) from any embodiments described herein.

Clause 1. A method comprising:
acquiring, using an ophthalmic imaging device, one or more diagnostic images of an eye of a patient;
applying one or more image processing algorithms to the one or more diagnostic images to determine a plurality of measurements corresponding to a plurality of anatomical parameters of the eye;
accessing patient suitability information for a prospective ophthalmic procedure, wherein the patient suitability information comprises a set of values corresponding to a subset of the plurality of anatomical parameters;
generating a suitability assessment for performing the prospective ophthalmic procedure on the eye, wherein generating the suitability assessment comprises comparing a subset of the plurality of measurements, corresponding to the subset of the plurality of anatomical parameters, with the set of values of the patient suitability information; and
presenting information from the suitability assessment using a patient-oriented display.

Clause 2. The method of Clause 1, wherein presenting the information from the suitability assessment comprises:
displaying at least one diagnostic image of the one or more diagnostic images; and
visually referencing one or more anatomical parameters of the subset of the plurality of anatomical parameters to a corresponding one or more portions of the at least one diagnostic image.

Clause 3. The method of Clauses 1 or 2, wherein:
applying the one or more image processing algorithms comprises identifying a plurality of anatomical structures of the eye,
the plurality of measurements are based on the plurality of anatomical structures, and
visually referencing the one or more anatomical parameters comprises highlighting one or more of the anatomical structures within the one or more diagnostic images.

Clause 4. The method of any of Clauses 1 to 3, wherein the one or more image processing algorithms includes one or both of: a feature detection algorithm and a feature extraction algorithm.

Clause 5. The method of any of Clauses 1 to 4, further comprising:
receiving a user input indicating an interaction of the patient with the presented information from the suitability assessment; and
displaying, based on the user input, additional information related to the presented information using the patient-oriented display.

Clause 6. The method of any of Clauses 1 to 5, wherein presenting information from the suitability assessment further comprises:
displaying a graph of a first measurement of the plurality of measurements, corresponding to a first anatomical parameter of the subset of the plurality of anatomical parameters, relative to the values of the first anatomical parameter for a reference population.

Clause 7. The method of any of Clauses 1 to 6, further comprising:
displaying, based on the plurality of anatomical parameters, a first view of a reference image simulating a view of the eye after performing the prospective ophthalmic procedure.

Clause 8. The method of any of Clauses 1 to 7, wherein:
the first view corresponds to performing the prospective ophthalmic procedure according to a first parameter, and
the method further comprises:
displaying, based on the plurality of anatomical parameters, a second view of the reference image simulating a view of the eye after performing the prospective ophthalmic procedure according to a second parameter.

Clause 9. The method of any of Clauses 1 to 8, wherein:
the prospective ophthalmic procedure is a cataract surgical procedure,
the first parameter is a non-diffractive intraocular lens (IOL), and
the second parameter is a diffractive IOL.

Clause 10. A system comprising:
one or more processors;
one or more computer-readable storage media; and
program instructions stored on the one or more computer-readable storage media to cause the one or more processors to perform operations comprising:
acquiring, using an ophthalmic imaging device, one or more diagnostic images of an eye of a patient;
applying one or more image processing algorithms to the one or more diagnostic images to determine a plurality of measurements corresponding to a plurality of anatomical parameters of the eye;
accessing patient suitability information for a prospective ophthalmic procedure, wherein the patient suitability information includes a set of values corresponding to a subset of the plurality of anatomical parameters;
generating a suitability assessment for performing the prospective ophthalmic procedure on the eye, wherein generating the suitability assessment comprises comparing a subset of the plurality of measurements, corresponding to the subset of the plurality of anatomical parameters, with the set of values of the patient suitability information; and
presenting information from the suitability assessment using a patient-oriented display.

Clause 11. The system of Clause 10, wherein presenting the information from the suitability assessment comprises:
displaying at least one diagnostic image of the one or more diagnostic images; and
visually referencing one or more anatomical parameters of the subset of the plurality of anatomical parameters to a corresponding one or more portions of the at least one diagnostic image.

Clause 12. The system of Clauses 10 or 11, wherein:
applying the one or more image processing algorithms comprises identifying a plurality of anatomical structures of the eye,
the plurality of measurements are based on the plurality of anatomical structures, and
visually referencing the one or more anatomical parameters comprises highlighting one or more of the anatomical structures within the one or more diagnostic images.

Clause 13. The system of any of Clauses 10 to 12, wherein the one or more image processing algorithms includes one or both of: a feature detection algorithm and a feature extraction algorithm.

Clause 14. The system of any of Clauses 10 to 13, wherein presenting information from the suitability assessment further comprises:
displaying a graph of a first measurement of the plurality of measurements, corresponding to a first anatomical parameter of the subset of the plurality of anatomical parameters, relative to the values of the first anatomical parameter for a reference population.

Clause 15. The system of any of Clauses 10 to 14, the operations further comprising:
displaying, based on the plurality of anatomical parameters, a first view of a reference image simulating a view of the eye after performing the prospective ophthalmic procedure.

While the foregoing is directed to aspects of the present disclosure, other and further aspects of the disclosure may be devised without departing from the basic scope thereof.

What is claimed is:

1. A method comprising:
acquiring, using an ophthalmic imaging device, one or more diagnostic images of an eye of a patient;
applying one or more image processing algorithms to the one or more diagnostic images to determine a plurality of measurements corresponding to a plurality of anatomical parameters of the eye;
accessing patient suitability information for a prospective ophthalmic procedure, wherein the patient suitability information comprises a set of values corresponding to a subset of the plurality of anatomical parameters;
generating a suitability assessment for performing the prospective ophthalmic procedure on the eye, wherein generating the suitability assessment comprises comparing a subset of the plurality of measurements, corresponding to the subset of the plurality of anatomical parameters, with the set of values of the patient suitability information; and
presenting information from the suitability assessment using a patient-oriented display.

2. The method of claim 1, wherein presenting the information from the suitability assessment comprises:
displaying at least one diagnostic image of the one or more diagnostic images; and
visually referencing one or more anatomical parameters of the subset of the plurality of anatomical parameters to a corresponding one or more portions of the at least one diagnostic image.

3. The method of claim 1, wherein:
applying the one or more image processing algorithms comprises identifying a plurality of anatomical structures of the eye,
the plurality of measurements are based on the plurality of anatomical structures, and
visually referencing the one or more anatomical parameters comprises highlighting one or more of the anatomical structures within the one or more diagnostic images.

4. The method of claim 3, wherein the one or more image processing algorithms includes one or both of: a feature detection algorithm and a feature extraction algorithm.

5. The method of claim 1, further comprising:
receiving a user input indicating an interaction of the patient with the presented information from the suitability assessment; and
displaying, based on the user input, additional information related to the presented information using the patient-oriented display.

6. The method of claim 1, wherein presenting information from the suitability assessment further comprises:
displaying a graph of a first measurement of the plurality of measurements, corresponding to a first anatomical parameter of the subset of the plurality of anatomical parameters, relative to the values of the first anatomical parameter for a reference population.

7. The method of claim 1, further comprising:
displaying, based on the plurality of anatomical parameters, a first view of a reference image simulating a view of the eye after performing the prospective ophthalmic procedure.

8. The method of claim 7, wherein:
the first view corresponds to performing the prospective ophthalmic procedure according to a first parameter, and
the method further comprises:
displaying, based on the plurality of anatomical parameters, a second view of the reference image simulating a view of the eye after performing the prospective ophthalmic procedure according to a second parameter.

9. The method of claim 8, wherein:
the prospective ophthalmic procedure is a cataract surgical procedure,
the first parameter is a non-diffractive intraocular lens (IOL), and
the second parameter is a diffractive IOL.

10. A system comprising:
one or more processors;
one or more computer-readable storage media; and
program instructions stored on the one or more computer-readable storage media to cause the one or more processors to perform operations comprising:
acquiring, using an ophthalmic imaging device, one or more diagnostic images of an eye of a patient;
applying one or more image processing algorithms to the one or more diagnostic images to determine a plurality of measurements corresponding to a plurality of anatomical parameters of the eye;
accessing patient suitability information for a prospective ophthalmic procedure, wherein the patient suitability information includes a set of values corresponding to a subset of the plurality of anatomical parameters;
generating a suitability assessment for performing the prospective ophthalmic procedure on the eye, wherein generating the suitability assessment comprises comparing a subset of the plurality of measurements, corresponding to the subset of the plurality of anatomical parameters, with the set of values of the patient suitability information; and
presenting information from the suitability assessment using a patient-oriented display.

11. The system of claim 10, wherein presenting the information from the suitability assessment comprises:
displaying at least one diagnostic image of the one or more diagnostic images; and
visually referencing one or more anatomical parameters of the subset of the plurality of anatomical parameters to a corresponding one or more portions of the at least one diagnostic image.

12. The system of claim 10, wherein:
applying the one or more image processing algorithms comprises identifying a plurality of anatomical structures of the eye,
the plurality of measurements are based on the plurality of anatomical structures, and
visually referencing the one or more anatomical parameters comprises highlighting one or more of the anatomical structures within the one or more diagnostic images.

13. The system of claim 12, wherein the one or more image processing algorithms includes one or both of: a feature detection algorithm and a feature extraction algorithm.

14. The system of claim 10, wherein presenting information from the suitability assessment further comprises:
displaying a graph of a first measurement of the plurality of measurements, corresponding to a first anatomical parameter of the subset of the plurality of anatomical parameters, relative to the values of the first anatomical parameter for a reference population.

15. The system of claim 10, the operations further comprising:
displaying, based on the plurality of anatomical parameters, a first view of a reference image simulating a view of the eye after performing the prospective ophthalmic procedure.
